(19) United States
(12) Patent Application Publication
(10) Pub. No.: US 2023/0295558 A1
(43) Pub. Date: Sep. 21, 2023

(54) METHOD TO SELECTIVELY PRODUCE METABOLITES FROM MICROALGAE OF THE GENUS GALDIERIA

(71) Applicant: PONTIFICIA UNIVERSIDAD JAVERIANA, Bogotá (CO)

(72) Inventors: Sandra BAENA GARZÓN, Bogotá (CO); Gina LÓPEZ RAMIREZ, Bogotá (CO)

(21) Appl. No.: 18/006,544

(22) PCT Filed: Jul. 16, 2021
(86) PCT No.: PCT/IB2021/056460

§ 371 Date: Jan. 23, 2023

(30) Foreign Application Priority Data
Jul. 24, 2020 (CO) NC2020/0009174

(51) Int. Cl. C12N 1/12; C12N 1/02; C12P 7/64; C09B 61/00

(52) U.S. Cl. CPC C12N 1/125; C09B 61/00; C12N 1/02; C12P 7/64; C12N 2500/12; C12N 2500/74; C12R 2001/89

(57) ABSTRACT

The present invention relates to a method to selectively produce metabolites from microalgae Galdieria sp. The method of the present invention comprises multiplying the biomass of the microalgae in mixotrophic and/or heterotrophic conditions with the presence of salts, separating the biomass, washing the biomass and inducing the production of metabolites, where the culture conditions of the metabolite production stage depend on the molecule that is desired to be obtained.

### FIELD OF THE INVENTION

The present development is related to the area of biotechnology, more specifically to the cultivation of microalgae. In particular, the present invention discloses a method to selectively modulate the production of metabolites from extremophilic microalgae of Galdieria sp.

### BACKGROUND OF THE INVENTION

The production of metabolites with industrial value such as proteins, polysaccharides, vitamins, lipids and pigments from microalgae has become important in recent years. Mainly, the metabolic versatility of microalgae and the possibility of producing metabolites on a large scale from them has motivated advances in the optimization of microalgae cultivation conditions and obtaining by-products related to them.

Within the technique, microalgae of the genera Arthrospira Dunaliella, Haematococcus and Chlorella have been exploited on a large scale. However, studies on the potential to obtain metabolites of extremophilic microalgae of Galdieria sp. are still in very early stages.

In relation to methods of microalgae culture, in the state of the art there are known procedures that allow to potentiate the production of biomass or a specific metabolite from a species of microalgae. For example, patent document WO2012/100583 relates to a method for increasing microalgae biomass under heterotrophic conditions.

On the other hand, the document WO2015/168458 describes a method to potentiate the production of biomass, proteins and lipids from microalgae under heterotrophic conditions.

However, there are no known methods in the art to selectively produce metabolites from Galdieria sp. microalgae, to thus exploit its metabolic versatility.

Based on the above, there is a need in the art to develop a culture method of extremophilic microalgae of Galdieria sp. to allow the selective production of metabolites from these microalgae.

### BRIEF DESCRIPTION OF THE INVENTION

The development revealed here is a method that allows to selectively produce metabolites of industrial interest from extremophilic microalgae Galdieria sp. The method of this disclosure comprises:
a) multiplication of the biomass of microalgae under mixotrophic and/or heterotrophic conditions;
b) separation of the biomass from the culture medium;
c) washing the biomass;
d) inducing the production of metabolites;
   where the culture medium in stage a) comprises NaCl and an organic source of nitrogen, and the stage is carried out at a temperature between 30 and 60° C.;
   where stage d) for induction of metabolites takes place in the presence of NaCl, at a temperature between 18 and 65° C., an acidic pH, and in the absence of carbon sources; and
   where specific culture conditions of stage d) are modified according to the metabolite to be produced.

Advantageously, the method of the present invention provides a strategy to potentiate the metabolic versatility of microalgae of Galdieria sp. In particular, by applying the method disclosed herein, it is possible to selectively produce a metabolite of interest from Galdieria sp. microalgae by modifying the culture conditions.

Also, and surprisingly, the investors have developed a methodology that allows the growth of extremophilic microalgae of Galdieria sp. and the obtaining of metabolites from the same in a culture close to mesophilic conditions, which facilitates their metabolic exploitation in large-scale production processes. Advantageously, the method described herein also allows the production of substances such as lipids and pigments from microalgae of Galdieria sp. (some not reported in this type of organisms), in addition to extracts with antioxidant and/or antimicrobial capacity.

### BRIEF DESCRIPTION OF THE FIGURES

FIG. 1 shows the growth kinetics of Galdieria sp. at different concentrations of NaCl, according to example 1 of the invention.

FIG. 2 shows cultures of Galdieria sp. at different concentrations of NaCl, according to example 1 of the invention.

FIG. 3 represents the proportions of neutral lipids, glycolipids, phospholipids and other lipid classes produced by Galdieria sp. according to example 3 of the invention.

FIG. 4 shows the proportions of pigments produced by Galdieria sp. according to example 4 of the invention.

FIG. 5 represents the evaluation of the antimicrobial activity of total lipids obtained during the multifactorial strategy against the strains Escherichia coli, Pseudomonas aureginosa, Bacillus subtilis, Sthaphylococcus aureus and Candida albicans, according to example 5 of the invention.

### DETAILED DESCRIPTION OF THE INVENTION

The present invention relates to a method to selectively produce metabolites of industrial interest from Galdieria sp. microalgae, wherein the method comprises:
a) multiplying the biomass of microalgae under mixotrophic and/or heterotrophic conditions;
b) separating the biomass from the culture medium;
c) washing the biomass;
d) inducing the production of metabolites.

In this way, the method of the invention allows to potentiate the production of a metabolite of interest from Galdieria sp. microalgae by modifying the culture conditions.

In a preferred embodiment of the invention, in step a) the microalgae are maintained in mixotrophic and/or heterotrophic conditions; more specifically, in the absence and in the presence of light and a source of organic carbon.

In a preferred modality, light is at an intensity between 500 and 1600 Ix; more preferably between 800 and 1400 Ix. In one embodiment, the intensity of light is 1100 Ix.

Preferably, the source of organic carbon is a sugar; preferably the sugar is selected from: sucrose, galactose, glucose, xylose, mannitol, sorbitol, trehalose and arabinose; more preferably the source of organic carbon is glucose at concentrations between 5 and 50 mM; more preferably between 15 and 35 mM. In one embodiment the glucose concentration is 25 mM.

According to one embodiment of the invention, stage a) is carried out at a temperature between 35 and 55° C., more preferably the temperature is maintained between 40 and 50° C. In one embodiment the temperature is maintained at 45° C.

According to a preferred embodiment of the invention, the culture medium in stage a) comprises NaCl in a concentration between 0 and 150 g/L. Preferably, the concentration of NaCl is between 20 and 100 g/L; more preferably between 30 and 80 g/L. In preferred embodiments the concentration of NaCl is 40 to 60 g/L. In one embodiment the concentration of NaCl is 50 g/L.

In one embodiment of the invention, the culture medium in stage a) is supplemented with an organic source of nitrogen. Preferably, the organic source of nitrogen is selected from the group comprising: yeast, yeast extract, peptone, casamino acids and tryptone. More specifically, the organic source of nitrogen is yeast extract. Preferably, yeast extract is supplemented in stage a) at a concentration between 0.1 and 2 g/L; more preferably between 0.9 and 1.5 g/L. In one embodiment the concentration of yeast extract is 0.5 g/L.

According to the present development, the culture medium in stage a) is supplemented with an inorganic source of nitrogen. In a preferred modality, the inorganic source of nitrogen can be selected from the group that comprises: sodium nitrate NaNO₃, ammonium sulfate (NH₄)₂SO₄; preferably the source of inorganic nitrogen is ammonium sulfate (NH₄)₂SO₄ at a concentration between 0.1 and 2 g/L; more preferably between 0.9 and 1.5 g/L. In one embodiment of the invention, ammonium sulfate (NH₄)₂SO₄ in stage a) is supplemented at a concentration of approximately 1.3 g/L.

According to one embodiment of the invention, the culture medium in stage a) is maintained in acidic conditions. Preferably, the pH in stage a) is maintained at a pH between 1.5 and 6.5; particularly between 2 and 3. In one embodiment the pH is maintained at approximately 2.5.

According to a modality of the invention, stage a) is carried out for a period between 3 and 9 days; specifically between 4 and 7 days. In one embodiment the stage lasts 5 days.

After the multiplication of the microalgae biomass in stage (a), the biomass is separated from the culture medium by any phase separation procedure, according to stage (b). Phase separation methods may comprise, but are not limited to: filtration, vacuum filtration, centrifugation, decantation, among others.

Once the biomass has been separated from the microalgae, it is washed at least once with an isotonic solution to remove the remaining medium, according to stage c) of the method.

Then, the production of metabolites is induced, according to stage d) of the method disclosed herein.

In a preferred modality of the invention, the induction of metabolites of stage d) occurs in a culture medium comprising NaCl in concentrations ranging from 15 g/L to 125 g/L; more specifically from 20 g/L to 115 g/L.

According to the present invention, the induction of metabolites in stage d) occurs at a temperature between 30 and 60° C.; more specifically between 28 and 55° C.

According to one embodiment of the invention, stage d), the stage of induction of metabolites, is carried out in a culture medium with pH lower than 7. Preferably, the pH in stage d) is between 2 and 5. In a more preferred embodiment, the pH is maintained at approximately 2.5.

According to the present development, the culture medium in stage d) is supplemented with an inorganic source of nitrogen. In a preferred modality, the inorganic source of nitrogen can be selected from the group that comprises: sodium nitrate NaNO₃, ammonium sulfate (NH₄)₂SO₄; preferably the source of inorganic nitrogen is ammonium sulfate (NH₄)₂SO₄ at a concentration between 0.05 and 2 g/L; more preferably between 0.1 and 1.5 g/L. In a preferred embodiment, the concentration of ammonium sulfate (NH₄)₂SO₄ in step d) is approximately 70% to 75% lower than that used in stage a) of the method.

Preferably, step d) is carried out at a temperature between 18 and 60° C., preferably between 25 and 50° C. Likewise, the culture medium in which the induction of the metabolite occurs is supplemented with NaCl at a concentration between 20 and 100 g/L, preferably between 50 and 100 g/L. According to this preferred modality of the invention, the pH of the culture medium is lower than 7, preferably the pH is between 2 and 4. According to this modality of the invention, the metabolites produced are preferably lipids. According to this modality of the invention polyunsaturated fatty acids are obtained, such as linoleic and α-linolenic acid, eicosapentaenoic acid; monounsaturated and saturated fatty acids.

In another preferred modality of the invention, stage d) is carried out at a temperature between 15 and 35° C., preferably between 20 and 30° C. In one embodiment the temperature is maintained at 25° C. Likewise, the culture medium in which the induction of the metabolite occurs is supplemented with NaCl at a concentration between 40 and 60, preferably between 45 and 55 g/L. In one embodiment of the invention the concentration of NaCl is 50 g/L. According to this preferred modality of the invention, the pH of the culture medium is lower than 7, preferably the pH is between 2 and 3. In one embodiment the pH is maintained at 2.5. According to this modality of the present invention, the induction of metabolites preferably takes place in the presence of light radiation; where light is provided in an intensity between 500 and 1600 Ix; more preferably between 900 and 1400 Ix. In one embodiment the light radiation is 1100 Ix. According to this modality of the invention, the metabolites produced are preferably pigments.

### EXAMPLES

The following examples illustrate a preferred embodiment of the present invention. However, it must be understood that these are not limiting, according to the knowledge of a person moderately versed in the matter.

### Example 1: Obtaining the Biomass of the Microalgae

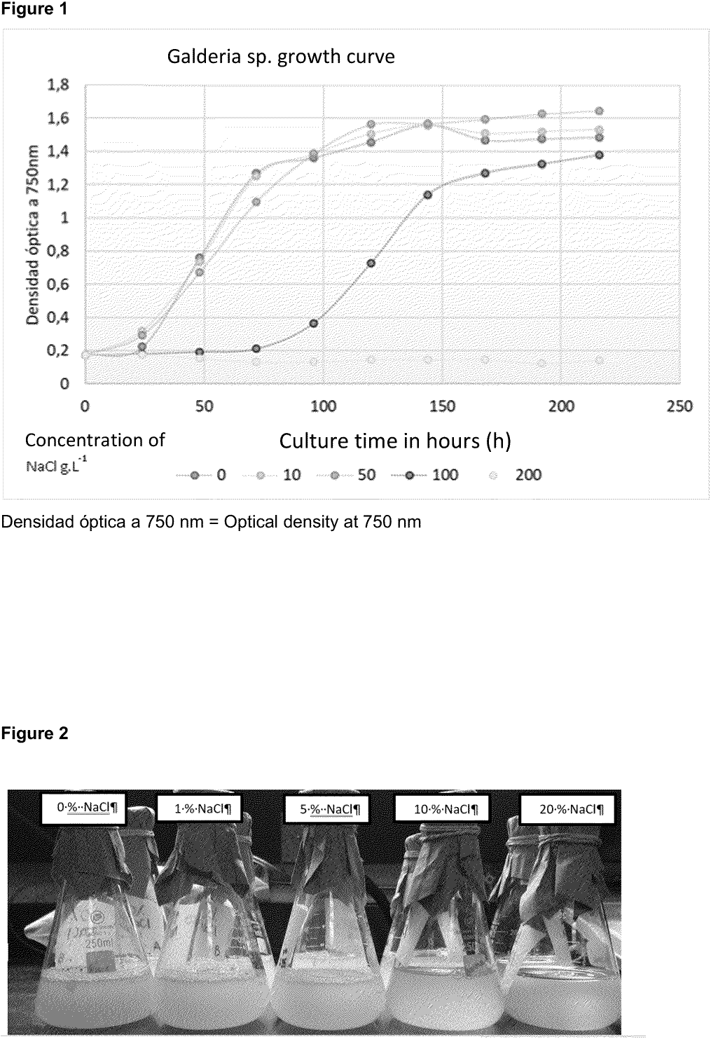

Culture conditions were optimized to increase the biomass of Galdieria sp. in heterotrophic conditions. For this, different concentrations of NaCl (0, 10, 50, 100 and 200 g/L) were evaluated, finding that at 50 g/L (5%) higher biomass productivity was obtained under heterotrophic conditions, as shown in FIGS. 1 and 2.

Having found the optimal concentration of NaCl to increase the biomass of Galdieria sp., the microalgae were cultured under a multiple-stage strategy, where in the biomass growth stage the culture conditions were: about 25 mM of glucose, at a pH of approximately 2.5, and about 0.5 g/L of yeast extract, with approximately 1.3 g/L (NH₄)₂SO₄ and 50 g/L of NaCl, under stirring at 190 rpm, in the presence of light (1100 Ix), obtaining a biomass productivity of approximately 337.44 mg.L⁻¹.d⁻¹.

### Example 2: General Experimental Conditions for Production of Metabolites From Galdieria sp.

After 6 days of cultivating the microalgae of Galdieria sp. in a biomass multiplication stage (according to stage 1 of the method disclosed herein), the microalgae were separated from the remaining culture medium by means of a centrifugation process at approximately 6000 g and were washed three times with saline (0.85%).

Subsequently, different conditions of light, pH, temperature and salinity (concentration of NaCl) were evaluated for the production of lipids and pigments from the microalgae of Galdieria sp. The treatments evaluated are summarized in Table 1.

TABLE 1

| Treatment | Light (lx) | pH | T (°C) | NaCl (%) |
|---|---|---|---|---|
| 1 | 1100 | 2.5 | 25 | 5 |
| 2 | 1100 | 2.5 | 50 | 5 |
| 3 | 1100 | 4.5 | 25 | 5 |
| 4 | 1100 | 4.5 | 50 | 5 |
| 5 | 1100 | 2.5 | 25 | 10 |
| 6 | 1100 | 2.5 | 50 | 10 |
| 7 | 1100 | 4.5 | 25 | 10 |
| 8 | 1100 | 4.5 | 50 | 10 |
| 9 | 0 | 2.5 | 25 | 5 |
| 10 | 0 | 4.5 | 25 | 5 |
| 11 | 0 | 2.5 | 25 | 10 |
| 12 | 0 | 4.5 | 25 | 10 |
| 13 | 0 | 2.5 | 50 | 5 |
| 14 | 0 | 4.5 | 50 | 5 |
| 15 | 0 | 2.5 | 50 | 10 |
| 16 | 0 | 4.5 | 50 | 10 |

### Example 3: Production of Lipids From Galdieria Sp

After 3 days of cultivation under the treatments established in example 2, the biomass of the microalgae of Galdieria sp. was lysed and the lipid content was extracted following a modified methodology of Bligh & Dyer¹. The fatty acid profile was evaluated by gas chromatography (CG [for the Spanish initials]) using an Rt-2560 column (Restek) and CG coupled to masses, according to the protocols described by Cavonius et al.² and González-Menéndez et al.³, respectively.

¹ Bligh, E. G. & Dyer, W. J. A rapid method of total lipid extraction and purification. Canadian Journal of Biochemistry and Physiology 37, 911-917, https://doi.org/10.1139/o59-099 (1959).

² Cavonius, L. R., Carlsson, N. G. & Undeland, I. Quantification of total fatty acids in microalgae: comparison of extraction and transesterification methods. Analytical and Bioanalytical Chemistry 406, 7313-7322, https://doi.org/10.1007/s00216-014-8155-3 (2014).

³ González-Menéndez, V. et al. Multicomponent Analysis of the Differential Induction of Secondary Metabolite Profiles in Fungal Endophytes. Molecules 21, 234, https://doi.org/10.3390/molecules21020234 (2016).

The results regarding lipid productivity and the percentage of the same with respect to biomass s are shown in Table 2.

TABLE 2

| Tr | LIGHT (Ix) | pH | T °C | NaCl (%) | Productivity biomass mg/L/d | % lipids | Productivity lipids mg/L/d |
|---|---|---|---|---|---|---|---|
| 1 | 1100 | 2.5 | 25 | 5 | 339.14 | 5.42 | 11.04 |
| 2 | 1100 | 2.5 | 50 | 5 | 291.56 | 5.46 | 9.55 |
| 3 | 1100 | 4.5 | 25 | 5 | 364.61 | 5.12 | 11.21 |
| 4 | 1100 | 4.5 | 50 | 5 | 286.17 | 5.24 | 8.99 |
| 5 | 1100 | 2.5 | 25 | 10 | 346.72 | 4.75 | 9.89 |
| 6 | 1100 | 2.5 | 50 | 10 | 332.22 | 5.18 | 10.33 |
| 7 | 1100 | 4.5 | 25 | 10 | 398.89 | 5.65 | 13.53 |
| 8 | 1100 | 4.5 | 50 | 10 | 337.44 | 6.53 | 13.22 |
| 9 | 0 | 2.5 | 25 | 5 | 307.11 | 6.28 | 11.57 |
| 10 | 0 | 4.5 | 25 | 5 | 352.28 | 6.22 | 13.14 |
| 11 | 0 | 2.5 | 25 | 10 | 377.83 | 6.53 | 14.79 |
| 12 | 0 | 4.5 | 25 | 10 | 370.72 | 5.74 | 12.76 |
| 13 | 0 | 2.5 | 50 | 5 | 227.58 | 7.33 | 10.89 |
| 14 | 0 | 4.5 | 50 | 5 | 314 | 6.53 | 12.29 |
| 15 | 0 | 2.5 | 50 | 10 | 325.07 | 5.95 | 11.6 |
| 16 | 0 | 4.5 | 50 | 10 | 359.83 | 5.73 | 12.36 |

Treatments 11 and 13 were found to provide the highest lipid productivity (mg/L/d) and the highest percentage of lipids, respectively.

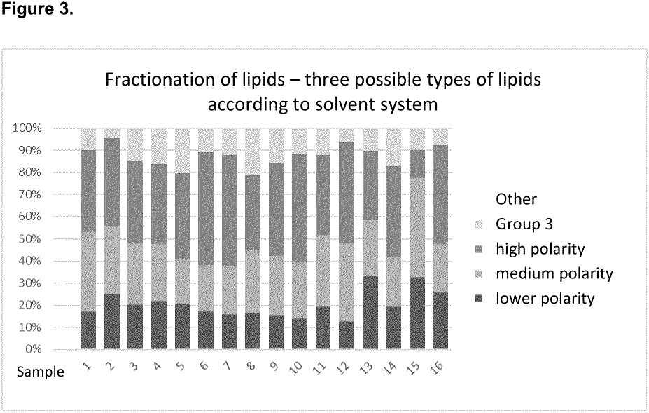

Additionally, and as shown in FIG. 3, the proportion of neutral lipids, glycolipids and phospholipids in each one of the treatments was analyzed, finding that the microalgae of Galdieria sp. have an important metabolic versatility.

Finally, it was possible to observe that the polyunsaturated fatty acids with the highest presence in the lipid fractions were linoleic (C_{18:2}) and linolenic (C_{18:3}), and in trace concentrations polyunsaturated fatty acids of very long chain such as arachidonic (C_{20:4}), eicosapentaenoic (C_{20:5}) and docosahexaenoic (C_{22:5}). Being for the first time reported for organisms of the genus Galdieria.

### Example 4: Production of Pigments From Galdieria Sp.

After 3 days of cultivation under the treatments established in example 2, pigments were extracted from the biomass of Galdieria sp., following the methodology of Asker⁴. Subsequently, the amount of chlorophyll a, chlorophyll b, carotenoids and phycocyanin (µ/mL or µ/mg) was quantified according to the methodology of Feller et al., 2018⁵. The pigment content was then analyzed by HPLC-DAD).

⁴ Asker, D. High throughput screening and profiling of high-value carotenoids from a wide diversity of bacteria in surface seawater. Food Chemistry, 261, 103-111, https://doi.org/10.1016/j.foodchem.2018.03.109 (2018).

⁵ Feller, R., Matos, Â. P., Mazzutti, S., Moecke, E. H.S., Tres, M. V., Derner, R. B., Oliveira, J.V., Junior, A.F. Polyunsaturated ω-3 and ω-6 fatty acids, total carotenoids and antioxidant activity of three marine microalgae extracts obtained by supercritical CO₂ and subcritical n-butane. The Journal of Supercritical Fluids. 133, 437-443. https://doi.org/10.1016;j.supflu.2017.11.015 (2018).

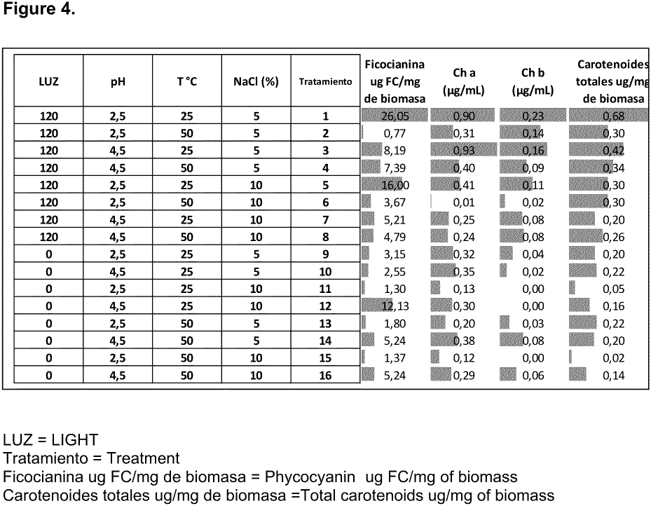

It was found that the greatest amount of pigments (chlorophyll a, chlorophyll b, carotenoids and phycocyanin), was achieved under conditions of light at a temperature of 25° C. and with a medium comprising NaCl at concentrations of approximately 5%, while the pH was maintained at 2.5. It must be noted that in this treatment the most abundant pigment was phycocyanin, as shown in FIG. 4.

### Example 5: Evaluation of Antimicrobial, Antioxidant and Cytotoxic Properties of Microalgae Extracts

Finally, an evaluation was made of the antioxidant and antimicrobial capacity of total lipids and fractions possibly rich in neutral lipids (fraction I), glycolipids (fraction II), and phospholipids (fraction III), of each one of the treatments established in example 2.

In particular, antioxidant activity was evaluated by means of the DPPH technique (2,2-diphenyl-1-picrylhydrazyl). The greatest antioxidant capacity was found in that fraction rich in neutral lipids (Fraction I), where the treatments with greater activity were 4, 5, 6, 12, with an activity around 85% of inhibition or amount of the DPPH radical neutralized by the extract. From the fraction rich in glycolipids, the best results were obtained with treatments 4, 5 and 12. The results are summarized in Table 3.

TABLE 3.

| | | | | | % of Antioxidant activity per DPPH | | | |
|---|---|---|---|---|---|---|---|---|
| Tr | LIGHT | pH | T °C | NaCl (%) | Total lipid | Fraction I | Fraction II | Fraction III |
| 1 | 1100 | 2.5 | 25 | 5 | 17.70 | 73.92 | 12.24 | 8.71 |
| 2 | 1100 | 2.5 | 50 | 5 | 8.85 | 69.22 | 8.22 | 10.42 |
| 3 | 1100 | 4.5 | 25 | 5 | 13.26 | 87.06 | 17.13 | 8.71 |
| 4 | 1100 | 4.5 | 50 | 5 | 17.07 | 88.63 | 51.75 | 9.28 |
| 5 | 1100 | 2.5 | 25 | 10 | 9.74 | 89.25 | 38.25 | 12.33 |
| 6 | 1100 | 2.5 | 50 | 10 | 10.75 | 88.84 | 17.89 | 12.72 |
| 7 | 1100 | 4.5 | 25 | 10 | 13.76 | 69.57 | 15.09 | 9.00 |
| 8 | 1100 | 4.5 | 50 | 10 | 11.82 | 11.79 | 11.93 | 7.83 |
| 9 | 0 | 2.5 | 25 | 5 | 1.57 | 40.68 | 6.44 | 5.13 |
| 10 | 0 | 4.5 | 25 | 5 | 3.33 | 86.12 | 11.45 | 10.06 |
| 11 | 0 | 2.5 | 25 | 10 | 7.52 | 70.72 | 16.64 | 9.27 |
| 12 | 0 | 4.5 | 25 | 10 | 7.52 | 88.71 | 31.84 | 8.88 |
| 13 | 0 | 2.5 | 50 | 5 | 0.00 | 79.03 | 13.82 | 11.98 |
| 14 | 0 | 4.5 | 50 | 5 | 5.52 | 28.14 | 12.93 | 19.25 |
| 15 | 0 | 2.5 | 50 | 10 | 13.38 | 43.55 | 8.62 | 7.66 |
| 16 | 0 | 4.5 | 50 | 10 | 14.26 | 47.86 | 13.29 | 11.59 |

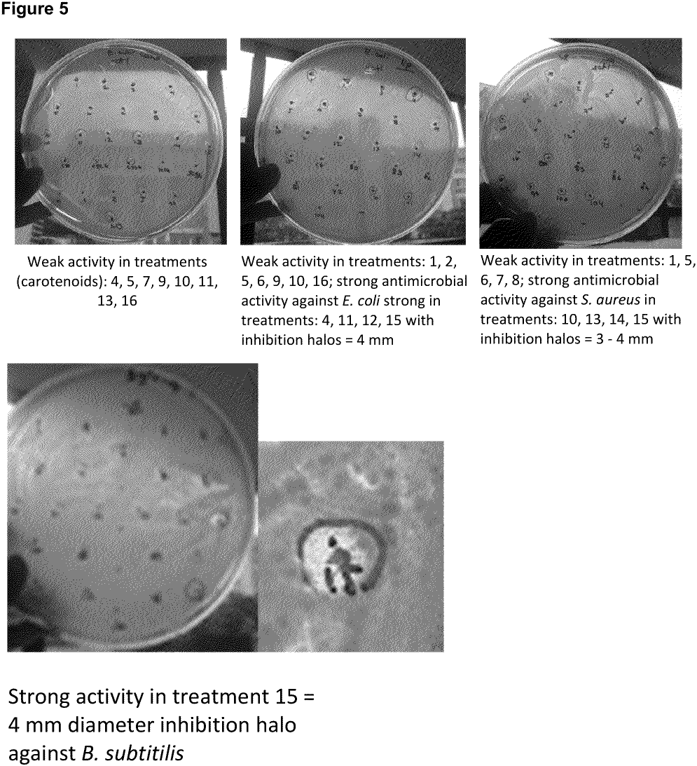

On the other hand, the antimicrobial activity of total lipids and fractions obtained from the culture of Galdieria sp. under different experimental conditions was estimated. For this, antibiograms were performed against the species: Escherichia coli, Pseudomonas aureginosa, Bacillus subtilis, Sthaphylococcus aureus and Candida albicans. The results are presented in FIG. 5, where it is possible to observe that some of the fractions presented activity for both Gram-negative and Gram-positive bacteria and yeasts. In addition, it was found that the total lipids of treatment 4 managed to counteract the growth of the different microorganisms evaluated (FIG. 5).

1. A method to selectively produce metabolites from microalgae of Galdieria sp., with the method comprising:
a) multiplication of microalgae biomass under mixotrophic and/or heterotrophic conditions;
b) separation of the biomass from the culture medium;
c) washing of the biomass; and
d) induction of the production of metabolites;
wherein the culture medium in stage a) comprises NaCl at a concentration between 0 and 150 g/L and an organic source of nitrogen, and the stage is carried out at a temperature between 30 and 60° C.;
wherein stage d), induction of metabolites, is carried out at a concentration of NaCl between 15 and 125 g/L, a temperature between 18 and 65° C. and with a pH between 2 and 5, in the absence of carbon sources; and
wherein the specific culture conditions of stage d) depend on the metabolite to be produced.

2. The method of claim 1 wherein temperature of stage a) is maintained between 35 and 55° C.

3. The method of claim 1 wherein stage a) is carried out for a period between 3 and 9 days.

4. The method of claim 1 wherein in stage a) the microalgae growth medium is maintained at acidic pH.

5. The method according to claim 4 wherein the pH of the culture medium in stage a) is maintained at a pH between 1.5 and 6.5.

6. The method according to claim 1 wherein the organic source of nitrogen in stage a) is yeast extract in concentrations between 0.1 and 2 g/L.

7. The method of claim 1 wherein the culture medium in stage a) is further supplemented with a source of inorganic nitrogen.

8. The method of claim 7 wherein the source of inorganic nitrogen is ammonium sulfate (NH₄)₂SO₄) at a concentration between 0.5 and 3 g/L.

9. The method according to claim 1 wherein in stage d) the microalgae are grown in a medium with NaCl at a concentration between 20 and 110 g/L, at a pH between 2 and 4, at a temperature between 18 and 60° C. in the absence or in the presence of light.

10. The method according to claim 1 wherein in stage d) the microalgae are grown in a medium with NaCl at a concentration between 40 and 60 g/L, at a pH between 2 and 3, at a temperature between 20 and 30° C. in the presence of light.

11. The method according to claim 10, wherein the culture medium in stage d) is further supplemented with an inorganic source of nitrogen.

12. The method according to claim 9 wherein the main metabolites produced by the microalgae are lipids.

13. The method of claim 10 wherein the metabolites mainly produced are pigments.

14. The method according to claim 9, wherein the culture medium in stage d) is further supplemented with an inorganic source of nitrogen.
